(19) United States
(12) Patent Application Publication
(10) Pub. No.: US 2008/0112991 A1
(43) Pub. Date: May 15, 2008

(54) METHOD OF ENHANCING SKIN ABSORPTION OF NUTRIENTS AND ACTIVE AGENTS

(75) Inventors: KARIN GOLZ-BERNER, Monaco (MC); Bernd Walzel, Zurich (CH); Leonhard Zastrow, Monaco (MC)

Correspondence Address: AKERMAN SENTERFITT, P.O. BOX 3188, WEST PALM BEACH, FL, 33402-3188, US

(73) Assignee: Coty B.V., Haarlem (NL)

(21) Appl. No.: 12/016,034

(22) Filed: Jan. 17, 2008

Related U.S. Application Data
(62) Division of application No. 10/725,659, filed on Dec. 2, 2003.

(30) Foreign Application Priority Data
Dec. 4, 2002 (DE) DE 102 57 240.2

(51) Int. Cl. A61K 31/714; A61K 31/525; A61K 31/51; A61K 31/355; A61K 31/205; A61K 31/07; A61K 9/14

(52) U.S. Cl. 424/401; 424/489; 514/52; 514/276; 514/251; 514/458; 514/725; 514/554

(57) ABSTRACT

The present invention relates to a cosmetic composition which contains electric carriers apart from specific active agents and auxiliaries. According to the present invention, the composition contains 0.1 to 10% by weight of a cosmetically acceptable, solid electret material with a particle size of 0.05 to 100 μm, which electret material is to be brought in the electrete state and has an induced permanent dipole moment and a permanent electric field with a field strength of 500 to 10⁷ Vm⁻¹, as well as cosmetic carrier substances, auxiliaries, further active agents or a mixture thereof in an amount ranging up to 100% by weight.

### FIELD OF THE INVENTION

The present invention relates to a cosmetic composition containing electric carriers apart from specific active agents and auxiliaries.

### BACKGROUND OF THE INVENTION

DE 43 25 071 discloses, inter alia, cosmetic preparations which contain permanent magnetic single crystals and are intended to stimulate micro-circulation in the skin.

WO 99/18892 describes bio-polymers, in particular alpha-helical polypeptides with electret properties, which may be used for improving tissue growth or for transplantations.

It is also known to use certain electrically polarised materials as filter materials in air purification.

### SUMMARY OF THE INVENTION

The object of the present invention is to improve the absorbing capacity of skin cells with regard to the supply of nutrients and active agents.

According to the present invention, the cosmetic composition containing electric carriers is characterised in that it contains 0.1 to 10% by weight of a cosmetically acceptable, solid electret material with a particle size of 0.05 to 100 μm, which electret material has an induced permanent dipole moment and a permanent electric dipol field with a field strength of 500 to 10⁷ Vm⁻¹, and in that it further contains cosmetic carrier substances, auxiliaries, further active agents or a mixture thereof in an amount ranging up to 100% by weight, the percentage data being relative to the total weight of the composition.

Preferred electrets have a dipol filed with a field strength of 10⁵ to 10⁷ Vm⁻¹.

Electrets are generally understood to be materials which have a permanent opposed electric charge at two opposing surfaces, thereby forming a permanent electric dipole surrounded by an electric field (dipol field). Certain natural electrets, such as tourmaline, have been known for a long time. However, it is also possible to impart electret properties to a number of materials by external influence. This may be a thermal influence, i.e. heating the materials above the Curie temperature and exposing them to an electric field in this heated state. The dipoles orienting themselves in the field are frozen by way of cooling. The state of non-equilibrium achieved in this way returns to a state of equilibrium after a certain relaxation time, which for electrets is in the range of years. The same states may also be achieved through photoelectric influence.

The field strengths of electrets are normally measured by means of high-impedance voltmeters by way of a simple measurement of the compensating voltage while simultaneously recording the distance between the material and the electrode and in dependence of the number of particles involved.

Both organic and inorganic materials may be brought into the electret state. Polarisation may be an atomic polarisation, a dipole polarisation, an interfacial polarisation, a space-charge polarisation or a polarisation by charge transfer. Dipole polarisation, which is essentially to be taken into account in this connection, is e.g. the case with certain polymers, such as polymerised fluorocarbons, or glasses, ceramics or glass ceramics which are brought into the electret state by means of heating, exposure of the material to an electric field and cooling, with the obtained dipole moment being preserved also during the subsequent comminution of the material.

The dipole moment given to the materials by that proceeding which herein shall be termed “induced permanent electric dipole moment” is clearly above the known dipole moment of materials which were not induced. The dipole moment of many compounds is known (e.g. Handbook of Chemistry and Physics, 70^{th} edition, Part E, p. 59 ff.; or McClellan, Tables of Experimental Dipole Moments, San Francisco: Freeman 1963) or may e.g. be determined by measuring the material's dielectric constant and density at different temperatures and using the Debye-Clausius-Mosotti equation.

For use in cosmetics, the electret material is comminuted until a particle size of 0.05 to 100 μm, preferably 3 to 80 μm, is achieved.

Preferred electret materials which are to be brought in the electrete state are polymerised fluorocarbons selected from the group consisting of polytetrafluoroethylene (PTFE), fluorinated ethylene propylene (FEP), polyvinylidene fluoride (PVDF), amorphous fluoropolymer (AF) and mixtures thereof.

Preferred ceramics or glass ceramics which are to be brought in the electrete state are those containing oxidic base materials, selected from the group consisting of zirconium oxide, titanium oxide, magnesium oxide, lithium oxide, calcium oxide, silicon dioxide, barium oxide and mixtures thereof.

It has been found that the cosmetic composition according to the present invention distinctly improves the absorbing capacity of skin cells with regard to the supply of nutrients and active agents.

A preferred range for the electret material in the cosmetic composition is 0.1-8% by weight, especially 0.1-5% by weight.

In a preferred embodiment of the present invention, the cosmetic composition contains the electret material, preferably polytetrafluoroethylene, and at least 0.1% by weight, preferably 0.5-2% by weight, relative to the overall composition, Vitamin A or a Vitamin A compound. In comparative tests, it has been found that in the presence of the electret material the Vitamin A absorption through the skin improves by about 40 to 70% within the same period of 0-4 hours.

In another preferred embodiment of the present invention, a similar improved absorption of active agents was found in respect of 0.1-2% by weight Vitamin E and derivatives thereof, whose absorption through the skin improved by about 30-50%.

In yet another preferred embodiment of the present invention, a similar improved absorption of active agents was found in respect of 0.1-3% by weight Creatine, whose absorption through the skin improved by about 25-55%.

Also the absorption of 0.1-3% by weight Vitamin B was found improved.

Preferred vitamin derivatives are e.g. tocopheryl acetate, tocopheryl palmitate, tocopheryl succinate, tocopheryl propionate, tocopheryl oleate, tocopheryl linolate, tocopheryl sorbate, and retinyl palmitate as well as other retinoids such as retinyl acetate, retinyl butyrate, retinyl propionate, retinyl octanoate, retinyl laurate, retinyl oleate, retinyl linolate.

It may be of advantage for the preparations according to the present invention that they contain in addition asymmetric lamellar aggregates, which aggregates consist of phospholipids and oxygen-loaded fluorocarbons or a fluorocarbon mixture and contain fluorocarbon in an amount ranging from 0.2 to 100% by weight/volume, wherein the phospholipid has a phosphatidylcholine content of more than 30 to 99% by weight and wherein the skin penetration of said aggregates depends upon the critical solubility temperature of the fluorocarbons. Said aggregates are oxygen carriers and facilitate the penetration of oxygen into the skin and thus a better supply of the skin with oxygen (for the use of asymmetric lamellar aggregates see also DE-B-42 21 255 or WO 94/00098).

Another active agent which may be contained in the cosmetic product according to the present invention are finely distributed hard magnetic single-range particles (single crystals) with a high coercive force of 3,000 to 5,000 oersteds and with particle sizes ranging from 50 to 1,200 nm, preferably 50 to 250 nm, with or without the above-mentioned asymmetric lamellar aggregates, which hard magnetic particles are in particular barium hexaferrites and/or strontium hexaferrites produced according to the glass crystallisation technology by the growing of single crystals from a quenched glass melt (see WO 95/03061, e.g. Example 2, 3 or 7; and WO 98/44895, e.g. Example 1C). Said single crystals improve micro-circulation thus enhancing still further the effect brought about by the particles according to the present invention.

The composition according to the present invention may advantageously contain further cosmetic active agents. They include e.g. emulsifiers, inorganic and organic sunscreens, scavengers, moisturisers, vitamins, enzymes, plant-based active agents, polymers, antioxidants, anti-inflammatory natural active agents; decomposition products of yeasts or plant materials obtained by a gentle ultrasonic decomposition process according to WO 94/13783; kaolin as well as kaolin modified with SiO₂ according to WO 94/17588.

Another preferred additive for the cosmetic product according to the present invention is an active agent preparation with a high radical protection factor containing a product obtained by extraction of the bark of Quebracho blanco and subsequent enzymatic hydrolysis, which product contains at least 90% by weight proanthocyanidine oligomers and not more than 10% by weight gallic acid and is contained in micro-capsules, as well as a silk-worm extract obtained by extraction, which extract contains the peptide cecropine, amino acids and a mixture of vitamins, and a non-ionic, cationic or anionic hydro-gel or a mixture of hydro-gels, and one or several phospholipids and water (e.g. WO 99/66881, active agent complex according to Example 1 or 2; WO 01/26617, active agent complex according to Example 1).

It is also advantageous to add suitable water-soluble and/or oil-soluble UVA or UVB filters or both to the compositions according to the present invention. Advantageous oil-soluble UVB filters include 4-aminobenzoic acid derivatives such as 4-(dimethylamino)-benzoic acid-(2-ethylhexyl)ester; esters of cinnamic acid such as 4-methoxycinnamic acid(2-ethylhexyl)ester; benzophenone derivatives such as 2-hydroxy-4-methoxybenzophenone; 3-benzylidene camphor derivatives such as 3-benzylidene camphor.

Preferred oil-soluble UV filters are Benzophenone-3, Butylmethoxybenzoylmethane, Octyl Methoxycinnamate, Octyl Salicylate, 4-Methylbenzylidene Camphor, Homosalate and Octyl Dimethyl PABA.

Water-soluble UVB filters are e.g. sulphonic acid derivatives of benzophenone or of 3-benzylidene camphor or salts such as the Na salt or the K salt of 2-phenylbenzimidazol-5-sulphonic acid.

UVA filters include dibenzoylmethane derivatives such as 1-phenyl-4-(4′-isopropylphenyl)propane-1,3-dion.

Inorganic pigments on the basis of metal oxides, such as TiO₂, SiO₂, ZnO, Fe₂O₃, ZrO₂, MnO, Al₂O₃, and mixtures thereof may also be used as sunscreens.

Particularly preferred additional active agents are oxygen-loaded asymmetric lamellar aggregates according to WO 94/00109 or WO 94/00098 and hard magnetic single crystals according to WO 95/03061.

The preparation according to the present invention further contains cosmetic auxiliaries and carrier substances as they are commonly used in such preparations, e.g. water, preservatives, colorants, pigments with a colouring effect, thickeners, fragrances, alcohol, polyols, esters, electrolytes, gel-forming agents, polar and non-polar oils, polymers, copolymers, emulsifiers, waxes, stabilisers.

The oils used for the present invention may be common cosmetic oils, such as a mineral oil; hydrogenated polyisobutylene; synthetic squalane or squalane made from natural products; cosmetic esters or ethers which may be branched or linear, saturated or unsaturated; vegetable oils; or mixtures of two or of several thereof.

Polyols are e.g. propylene glycol, dipropylene glycol, ethylene glycol, isoprene glycol, glycerine, butylene glycol, sorbitol and mixtures thereof. Polyol makes up 0.1 to about 20% by weight of the total composition.

A great number of compounds may normally be used as emollient, such as stearyl alcohol, glyceryl monoricinoleate, glyceryl monostearate, propan-1,2-diol, butan-1,3-diol, cetylic alcohol, isopropyl isostearate, stearic acid, isobutyl palmitate, isopropyl myristate, isopropyl palmitate, oleyl alcohol, isopropyl laurate, decyl oleate, octadecan-2-ol, isocetyl alcohol, cetyl palmitate, silicone oils such as dimethylpolysiloxane, polyethylene glycol, lanoline, cocoa butter, vegetable oils such as maize oil, cotton seed oil, olive oil, mineral oils, butyl myristate, palmitic acid etc.

Suitable gel-forming agents include Carbomer, Xanthan Gum, Carrageenan, Acacin, Guar Gum, Agar-agar, Alginates and Tyloses, Carboxymethyl Cellulose, Hydroxyethyl Cellulose, Quaternised Cellulose, Quaternised Guar, certain polyacrylates, polyvinyl alcohol, polyvinyl pyrrolidone (PVP), montmorillonite. Quaternised Guar, Carbomer and PVP are preferred.

The cosmetic compositions according to the present invention may e.g. be used in the form of sun creams, sun gels, after-sun products, day creams, night creams, masks, body lotions, cleansing milk, body powder, eye cosmetics, hair masks, hair conditioners, hair shampoos, shower gels, shower oils, bath oils and in products of decorative cosmetics such as deo sticks, perfume sticks, lipsticks, gels, eye shadows, compact products such as compact powder or compact wax, rouge, foundations, make-up, etc. Such products are manufactured in a way that is known to the person skilled in the art.

No specific conditions are necessary for the inclusion of the electrets into the cosmetic formulations. It is advantageous to incorporate the electret particles at the end and not at temperatures above 50° C.

Another object of the present invention is the use of electrets with a field strength of 500 to 10⁷ Vm⁻¹ of the dipol field in an amount ranging between 0.1 and 10% by weight and with a particle size of 0.05 to 100 μm, selected from polymerised fluorocarbons, polyethyleneterephthalate, polymethyl methacrylate, polyimides, polypropylene, polyethylene, polyurethanes, polyureas, ceramics, glasses, glass ceramics and mixtures thereof, all of which have been converted into an induced electret state, together with cosmetic carrier substances, auxiliaries, other active agents or a mixture thereof in an amount ranging up to 100% by weight in cosmetic creams, lotions, gels, powders and sticks.

Another object of the present invention is a cosmetic composition containing electric carriers which comprises 0.1 to 10% by weight of a cosmetically acceptable, solid electret material with a particle size of 0.05 to 100 μm, which electret material has an induced permanent dipole moment and a permanent electric dipole field with a field strength of 500 to 10⁷ Vm⁻¹, furthermore comprising cosmetic carrier substances, auxiliaries, further active agents or a mixture thereof in an amount ranging up to 100% by weight, the percentage data being relative to the total weight of the composition, prepared by heating of a non-ferromagnetic solid material to a temperature below its melting temperature but above its softening temperature, exposing of the solid material to an electric field of 1000 to 10⁷ V/m, spontaneous cooling the solid material, grinding of the produced electret material to a particle size of 0.05 to 100 μm and mixing into a cosmetic composition below 50° C. Temperatures over the softening point are preferred in case of use of polymers as non-ferromagnetic materials to produce the electret material as foil to get surface loading densities of up to 10⁻⁷ C/cm² on the foil surface.

The invention will be described below in greater detail by means of examples. All data are given in % by weight, unless stated otherwise.

### EXAMPLE 1

### Night Cream

Phas A

| Water | q.s. ad 100 |
|---|---|
| Glycerine | 2.0 |
| Propylene Glycol | 2.0 |
| Carbomers | 0.3 |

Phase B

| | |
|---|---|
| Glyceryl Stearate | 1.0 |
| C12-15 Alkyl Benzoate | 1.5 |
| Silicone-Dimethicone | 2.5 |

Phase C

| | |
|---|---|
| Triethanolamine | 0.3 |

Phase D

| | |
|---|---|
| Polytetrafluoroethylene 10-60 μm | 5.0 |
| Vitamin C | 1.0 |
| Vitamin B6 | 2.0 |
| Perfume | 0.5 |
| Preservative | 0.5 |

After separate manufacture, phases A and B are heated up to approx. 75° C. while stirring, they are mixed with each other and the mixture is homogenised for 20 minutes at about 3,000 rpm. Thereafter, phase C is added, the mixture is homogenised for 5 minutes and cooled down to approx. 45° C. Phase D is added while the cooling-down and stirring continues and the mixture is homogenised for 20 minutes at 30° C. PTFE has a dipol moment of about 10⁻¹⁹ C.m.

### EXAMPLE 2

### Day Fluid

Phase A

| | |
|---|---|
| Cetyl Palmitate | 1.0 |
| Cetearyl Isononanoate | 1.8 |
| Squalane | 2.9 |

Phase B

| | |
|---|---|
| Water | q.s. ad 100 |
| Sodium Citrate | 0.1 |
| Glycerine | 2.0 |
| Polyacrylamide | 0.1 |

Phase C

| | |
|---|---|
| Fluorocarbonpolymer mixture¹ | 2.0 |
| Preservative | 0.5 |
| Perfume | 0.2 |
| Caffeines | 0.3 |

¹a mixture of 20% polytetrafluoroethylene, 30% fluorinated ethylene propylene, 30% polyvinylidene fluoride and 20% amorphous fluoropolymer (particle size 50-90 μm; dipole moment about 10⁻¹⁶ C.m).

The further manufacture of the cosmetic composition takes place according to Example 1.

### EXAMPLE 3

### Body Lotion I

Phase A

| | |
|---|---|
| PEG 100 Stearate | 3.5 |
| C12-15 Alkyl Benzoate | 1.0 |
| Shea Butter Fruit | 2.5 |

Phase B

| | |
|---|---|
| Water | q.s. ad 100 |
| Carbomer | 0.2 |
| Glycerine | 2.0 |

Phase C

| | |
|---|---|
| Triethanolamine | 0.2 |

Phas D

| | |
|---|---|
| Polytetrafluoroethylene 0.5-40 μm | 0.15 |
| Vitamin A | 0.1 |
| Perfume | 0.8 |
| Preservative | 0.5 |

### EXAMPLES 4 AND 5

### Body Lotions II and III

A lotion was prepared in accordance with Example 3 with the exception that 0.5% of an active agent complex according to Example 1 of WO 01/26617 was used instead of Vitamin A, which complex consisted of bark extract of Quebracho blanco, silk-worm extract, gel, phospholipids, superoxide dismutase from yeast decomposition product, cyclodextrines and water (Example 4).

In addition, a body lotion III was produced containing 0.1% Vitamin A and 0.5% of the aforementioned active agent complex (Example 5). Dipol moments about 10⁻²¹ C.m.

### EXAMPLE 6

### Night and Day Cream I

Phase A

| Water | q.s. ad 100 |
|---|---|
| Glycerine | 1.5 |
| Propylene Glycol | 1.5 |
| Carbomer | 0.15 |

Phase B

| | |
|---|---|
| Glyceryl Stearate | 1.5 |
| Shea Butter Fruit | 3.5 |
| Silicone-Dimethicone | 2.0 |

Phase C

| | |
|---|---|
| Triethanolamine | 0.15 |

Phas D

| | |
|---|---|
| Polytetrafluoroethylene 10-60 μm | 8.0 |
| Aggregates containing O₂ | |
| according to WO 94/00098¹ | 10.0 |
| Vitamin B6 | 2.0 |
| Perfume | 0.2 |
| Preservative | 0.5 |

¹according to Example 1 of WO with about 60% perfluorocarbons (perfluorodecalin).

Processing was done in accordance with Example 1.

### EXAMPLE 7

### Night and Day Cream II

Phase A

| Water | q.s. ad 100 |
|---|---|
| Glycerine | 1.5 |
| Propylene Glycol | 1.5 |
| Carbomer | 0.15 |

Phase B

| | |
|---|---|
| Glyceryl Stearate | 1.5 |
| Shea Butter Fruit | 3.5 |
| Silicone-Dimethicone | 2.0 |

Phase C

| | |
|---|---|
| Triethanolamine | 0.15 |

Phase D

| | |
|---|---|
| Polytetrafluoroethylene 10-60 μm | 8.0 |
| Aggregates containing hard magnetic | |
| Barium Hexaferrite according to | |
| WO 95/03061¹ | 2.0 |
| Vitamin B6 | 2.0 |
| Perfume | 0.2 |
| Preservative | 0.5 |

¹according to Example 7 with perfluorodecalin.

Processing was done in accordance with Example 1.

1-11. (canceled)

12. A method for improving absorption of nutrients and/or active agents into the skin, the method comprising
adding electrets with an induced permanent dipole moment and a dipole field strength of 500 to 10⁷ Vm⁻¹ and with a particle size of 0.05 to 100 μm, selected from polymerised fluorocarbons, polyethylene-terephthalate, polymethyl methacrylate, polyimides, poly-propylene, polyethylene, polyurethanes, polyureas, ceramics, glasses, glass ceramics and mixtures thereof, all of which have been converted into an induced electret state, in an amount of 0.1 to 10% by weight based on the final total composition, to a cosmetic formulation comprising nutrients and/or active agents, and further comprising cosmetic carrier substances, auxiliaries, further active agents or a mixture thereof, and
applying said formulation to skin.

13. A method as in claim 12, wherein said formulation is in the form of a cosmetic cream, lotion, gel, powder or stick.

14. A method according to claim 12, wherein said nutrient or active agent is selected from the group consisting of Vitamin A, Vitamin B, Vitamin E, creatine, derivates thereof, und mixtures thereof.
